(19) United States
(12) Patent Application Publication
(10) Pub. No.: US 2006/0126859 A1
(43) Pub. Date: Jun. 15, 2006

(54) Sound system improving speech intelligibility

(76) Inventor: Claus Elberling, Hellerup (DK)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 10/543,416

(22) PCT Filed: Jan. 29, 2004
(86) PCT No.: PCT/DK04/00061

§ 371 Date: Sep. 7, 2005

(30) Foreign Application Priority Data
Jan. 31, 2003 (DK) PA 2003 00132

(51) Int. Cl. A61F 11/06; G10K 11/16

(52) U.S. Cl. 381/71.1

(57) ABSTRACT

The invention relates to a method and a device for improving speech intelligibility for a listener receiving a speech signal output through a transducer in a noisy environment, where in the speech signal prior to the output one or more parameters have been modified in a signal processor corresponding to what a speaking person would normally do when speaking in a noisy environment or when speaking clearly.

### AREA OF THE INVENTION

The invention relates to sound delivery systems, where a sound source is delivering a sound signal to a listener. More specifically the invention relates to a method for improving the intelligibility of the output signal in such sound delivery systems as well as a sound delivery system implementing the method.

### BACKGROUND OF THE INVENTION

In many situations a speech signal is output to a listener, where the listener is in a noisy environment and where the speech signal originates as a signal performed in a silent or at least less noisy environment than the location of the listener.

Examples of such situations include telephone communication situations, where one telephone device is located in a noisy environment and another is in a quiet environment, ATM dispensing situations and similar situations, where a voice instruction is given automatically or upon request and where the environment may be noisy.

The objective of the present invention is to provide a remedy for the noisy listening situations where a listener may have difficulties understanding a voice message spoken or recorded in quiet conditions.

Vocal effort signifies the way normal speakers adapt their speech to changes in background noise, acoustic environment or communication distance. Specifically, vocal effort provoked by changing background noise is often referred to as the Lombard reflex, -effect or -speech after the French ENT-doctor E. Lombard (Lombard, 1911—see also Sullivan, 1963).

Similarly, ‘clear speech’ signifies the way normal speakers may adapt their speech when they want to improve speech intelligibility in various acoustical backgrounds (Krause & Braida, 2002).

Speech spoken with different vocal efforts can perceptually be classified into being soft, normal, raised, loud or shouted. However, in the scientific literature other classification labelling can also be found.

Variation in vocal effort is physiologically associated with changes in the airflow through the glottis, in the movements of the vocal cords, in the muscles of the pharynx, and in the shape of the vocal tract (Holmberg et al., 1988 & 1995; Ladefoged, 1967; Schulman, 1989; Södersten et al., 1995).

Perceptual experiments have demonstrated that speech produced with increased vocal effort is more intelligible than normal speech (Summers et al., 1988). It thus appears that speakers attempt to maintain an almost constant level of speech intelligibility when the information becomes degraded by environmental noise.

The most salient feature of vocal effort is probably the changes in the all-over amplitude and spectral characteristics of the speech signal. Pearsons et al. (1978) first described this in detail for face to face communication in background noise and these results has later been included in the Speech Intelligibility Index—standard (ANSI, 1997). Pearsons et al. found that all-over speech level increases systematically about 0.6 dB/dB as a function of background noise level. However, a more significant effect was found at higher-frequencies (a spectral tilt) resulting in an increase of about 0.8 dB/dB in the 1-3 kHz area. Others have made similar qualitative findings (Childers & Lee, 1991; Granström & Nord, 1992; Gauffin & Sundberg, 1989; Liénard & Di Benedetto, 1999). Since most background noises are dominated by low frequency energy, the speech changes associated with vocal effort attempt to maintain the audibility of the high frequency speech elements even in adverse signal-to-noise ratios. Normally, speech information is highly redundant, so if audibility of the high frequency speech elements is maintained when communicating in background noise, adequate speech intelligibility will be ensured for people with normal hearing.

Besides the all-over amplitude and spectral changes described above, a series of other acoustic-phonemic features are also influenced by vocal effort. The following changes to increased vocal effort have been reported in the literature: decrease in rate of speaking (Hanley & Steer, 1949), increase of the pitch frequency, F₀, and of the first formant frequency, F₁, (Bond et al., 1989; Draegert, 1951; Junqua, 1993; Liénard & di Benedetto, 1999; Loren et al., 1986; Rastatter & Rivers, 1983; Summers et al., 1988), increase in vowel duration and decrease in consonant duration (Bonnot & Chevrie-Muller, 1991; Fónagy & Fónagy, 1966, Rostolland, 1982, Traunmüller & Eriksson, 2000), and decrease in consonant/vowel energy ratio (Fairbanks & Miron, 1957; Junqua, 1993).

Both acoustical and perceptual analysis suggests that the Lombard effect works differently in male and female speakers. This gender effect has been studied systematically by Junqua (1993).

In Summary

The following acoustic-phonetic speech features appear to be affected by vocal effort:
level
frequency spectrum
rate of speaking
pitch, F₀
formant frequency, F₁
vowel and consonant duration
consonant/vowel energy ratio
   and the observed changes are gender-specific.

### SUMMARY OF THE INVENTION

According to the invention the objective of the invention is achieved by means of the method as defined in claim 1.

By means of such modification of the output signal the intelligibility will be improved for the listener being in a noisy environment. Not all types of environmental noise will affect speech communication to the same extent. For example, a very low frequency noise signal will not affect the information in the speech signal (which is limited to frequencies above 100 Hz) although the sound level alone would indicate so. Therefore, not all noise types should activate a vocal effort processor as defined in claim 1 in the same way, and by monitoring parameters other than all-over sound level would guide the function of the vocal effort processor to an appropriate response to different noise types.

Preferably at least one between the following parameters of speech is modified: level, frequency spectrum, rate of speaking, pitch F₀, one or more formant frequencies F₁, F₂, . . . , vowel and consonant duration, consonant/vowel energy ratio.

According to the invention the objective of the invention is achieved by means of the sound delivery system as defined in claim 3.

By means of such modification of the output signal the intelligibility will be improved for the listener being in a noisy environment.

The invention will be described in more detail in the following description of embodiments, with reference to the drawings.

### BRIEF DESCRIPTION OF THE DRAWINGS

FIG. 1 is a schematic drawing showing an example of a sound delivery system where the invention may be implemented,

FIG. 2 is a schematic drawing showing a further example of a sound delivery system where the invention may be implemented.

### DESCRIPTION OF A PREFERRED EMBODIMENT

The embodiment is characterised by the transmitter and the receiver of a communication channel being located in two environments with different environmental background noise conditions. Thus conditions for producing speech in environment 1 and the conditions in environment 2 for listening to the speech will be different. If the speaker and listener were in the same environment, the speakers voice would adapt to the level of the background noise—the vocal effort would be activated—and this ensures that a normal hearing listener could understand what the speaker is saying.

However when the speaker and listener are not in the same environment, the background noise of environment 2 will not normally activate vocal effort with the speaker in environment 1. It is the idea of present invention to artificially produce the missing vocal effort, of the speaker in environment 1 so as to ease the understanding of the listener in environment 2.

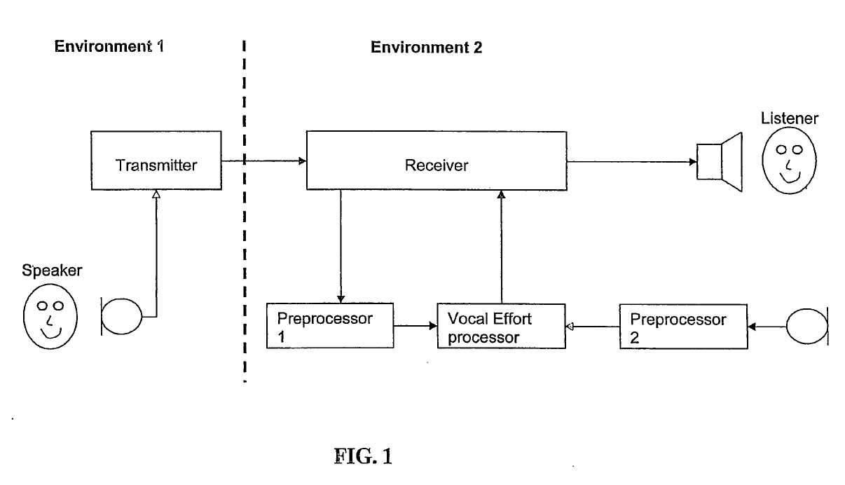

In the embodiment shown on FIG. 1 the sound is either picked up directly from the speaker, synthesised from text or other input or it is pre-recorded and stored for later use. At request or on-line the speech is then sent to environment 2, where the intended listener is located. The speech can be sent in the communication channel either as an analogue signal, a digital signal or as parameters of a speech or audio codec.

From the speech received by the receiver a number of parameters characterising the incoming speech signal is deduced by “Pre-processor 1”. These parameters are compared to a similar set deduced from environment 2 by pre-processor 2 in a vocal effort processor, which then adds vocal effort to the incoming speech signal if necessary. The parameters deduced by pre-processor 1 and 2 could be level, frequency tilt and long term spectrum, Voice Activity Detection (VAD) and Speech to Noise Ratio (SpNR).

Given the SpNR of the incoming signal (environment 1) and the SpNR of environment 2, it is possible to correct the incoming signal for the degree of lack of vocal effort, so that the listener in environment 2 more easily hears it.

The addition of vocal effort to the incoming signal can be done in several ways. A first order approach is to only correct for level and frequency spectrum. As a second order approach the duration and height of vowels and consonants can also be addressed. The addition of vocal effort can either be done directly in the vocal effort processor or in the receiver, as indicated by parameters sent from the vocal effort processor to the receiver.

For applications involving the first order approach the addition of the vocal effort could typically be performed in the vocal effort processor itself. For applications involving the second order approach, this typically involves the use of a speech or audio codec, so therefor it would be more straight forward to let the vocal effort processor modify the parameters of the incoming speech so that the receiver itself would resynthesize the speech with the vocal effort. This latter implementation approach makes the invention more computationally efficient, if implemented in digital technology and thus also more power efficient.

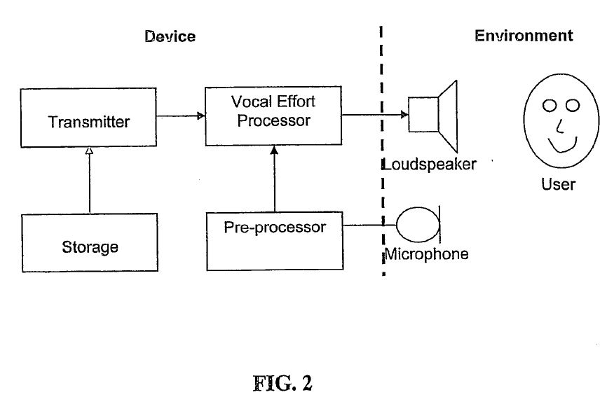

In a second preferred embodiment shown on FIG. 2 pre-recorded speech or parameters of speech, for instance for speech synthesis is stored in a storage means in a device, for instance a bank terminal, tourist information terminal or other devices placed in an environment in which ambient noise levels often are problematic. The speech or parameters of speech, for instance for speech synthesis stored in the storage means does not contain vocal effort. So if this is needed for proper communication in the environment, for instance due to a high level of ambient noise, it becomes difficult for the user of the device to understand the message from the device. It is the idea of the invention to artificially produce the missing vocal effort, of the speech from the device, so as to ease the understanding of the user.

From the signal received by the pre-processor (from the microphone) a number of parameters characterising the incoming signal is deduced by a pre-processor, as described in connection to the first example embodiment. These parameters are compared to predefined values or a set of rules, indicating when vocal effort is necessary. The vocal effort processor then adds vocal effort to the speech signal whenever it is necessary.

The speech can be sent to the transmitter either as an analogue signal, a digital signal or as parameters of a speech or audio codec. In the first two cases, the transmitter becomes a simple analogue or digital amplifier and in the last case the speech parameters are first used to synthesise a speech signal before it is amplified and sent to the vocal effort processor.

In an alternative embodiment—in stead of adding the vocal effort after the speech is recorded or synthesised, it could also be possible to store different versions of the speech or parameters for speech synthesis, which include different levels of vocal effort. These versions could then be used so that they match the ambient noise level, and the user then listens to a signal with the proper amount of vocal effort.

In another embodiment, the device uses online speech recognition to recognise the input from the user. The message from the device is then the response to what the user just said. In that connection, the device could use the information regarding the ambient noise level, and other parameters of the environment to decide how to recognise the speech. It is well known from the literature, that some features extracted from speech are more noise robust than others. So when no or little noise is present it is not necessary to perform speech recognition with a large feature set, only a subset of the feature set is used. However as the ambient noise increases in level or becomes more disturbing for the speech recogniser, a larger feature set, including more noise robust features of speech is used.

The embodiment shown on FIG. 1 could be implemented in a mobile phone. This could be done in a number of ways, including modification of the parameters of the synthesis filter, modification of the function of the de-emphasis filter or simply by adding a separate filter after the synthesis filter. The information necessary for estimating the speech to noise ratio, SpNR, in both environments, to be used for estimating a lack of vocal effort for one of the listeners, could be computed in the voice activity detection, VAD, part of the speech codec. In the VAD a substantial amount of the information needed to estimate the SpNR is already available, for instance in GSM-phones today. By adding to this an estimate of the modulation in the observed signal, an estimate of the SpNR. Since the addition of the vocal effort is only relevant when speech is present, the use of the VAD output can be used to turn the vocal effort processing on and off, as it is done for the speech codes in GSM-phones today.

The embodiment shown on FIG. 2 has been implemented on a stand-alone PC, equipped with a standard sound card, and a database of pre-recorded utterances stored in the storage shown on the figure. In this case, the transmitter is a simple decoder, capable of reading the encoded digitized utterances from the storage. Once a selected utterance is converted in the transmitter to a series of digital voice samples, the vocal effort processor processes the digital speech samples by means of a digital FIR-filter. The amount of amplification and spectral shape of the FIR-filter is controlled by the pre-processor. The pre-processor calculates an estimate of the L_{eq} of the digitized signal from the microphone in 6 octave bands with midband frequencies 0.25, 0.5, 1, 2, 4, 8 kHz. The estimate of the L_{eq} is continuously updated. By means of the L_{eq}'s which are interpreted as a coarse estimate of the ambient noise spectrum, the amount of vocal effort to apply to the speech signal is determined by means of a look-up table. The look-up table defines standard speech spectrum levels for different vocal effort, ranging from normal over raised and loud to shout. By calculating the difference between the ambient noise spectrum and the corresponding spectrum of speech at that ambient noise level, as defined by the look-up table, the gain and frequency spectrum of the FIR-filter of the vocal effort processor is calculated. Finally the calculated filter characteristics are applied to the FIR-filter of the vocal effort processor, which then changes the vocal effort of the pre-recorded voice utterances to match the ambient noise level.

The standard speech spectrum levels for different degrees of vocal effort, is listed in the table below.

TABLE 1
Octave band speech spectrum - frequencies
and standard speech spectra.

| | | |
|---|---|---|
| | Nominal | Standard speech spectrum level for |
| Band | midband | stated vocal effort, dB |

| No. | freq., Hz. | Normal | Raised | Loud | Shout |
|---|---|---|---|---|---|
| 1 | 250 | 34.75 | 38.98 | 41.55 | 42.50 |
| 2 | 500 | 34.27 | 40.15 | 44.85 | 49.24 |
| 3 | 1000 | 25.01 | 33.86 | 42.16 | 51.31 |
| 4 | 2000 | 17.32 | 25.32 | 34.39 | 44.32 |
| 5 | 4000 | 9.33 | 16.78 | 25.41 | 34.41 |
| 6 | 8000 | 1.13 | 5.07 | 11.39 | 20.72 |

Source: SII-procedure, ANSI S3.5 1997.

### REFERENCE LIST

ANSI S3.5 (1997). ‘Methods for calculation of the speech intelligibility index’. American National Standard.
Bond, Z. S., Moore, T. J. and Gable, B. (1989). ‘Acoustic-phonetic characteristics of speech produced in noise and while wearing an oxygen mask’. J. Acoust. Soc. Am. 85, 907-12.
Bonnot, J-F. P. and Chevrie-Muller, C. (1991). ‘Some effects of shouted and whispered conditions on temporal organization of speech’. J. Phonetics 19, 473-83.
Childers, D. G. and Lee, C. K. (1991). ‘Vocal quality factors: Analysis, synthesis, and perception’. J. Acoust. Soc. Am. 90, 2394-2410.
Draegert, G. L. (1951). ‘Relationships between voice variables and speech intelligibility in high noise levels’. Speech Monogr. 18, 272-78.
Fairbanks, G. and Miron, M. (1957). ‘Effects of vocal effort upon the consonant-vowel ratio within the syllable’. J. Acoust. Soc. Am. 29, 621-6.
Fónagy, I. and Fónagy, J. (1966). ‘Sound pressure level and duration’. Phonetica 15, 14-21.
Gauffin, J. and Sundberg, J. (1989). ‘Spectral correlates of glottal voice source waveform characteristics’. J. Speech Hear. Res. 32, 556-65.
Granström, B. and Nord, L. (1992). ‘Neglected dimensions in speech synthesis’. Speech Commun. 11, 459-62.
Hanley, T. D. and Steer, M. D. (1949). ‘Effect of level of distracting noise upon speaking rate, duration and intensity’. J. Speech Hear. Disord. 14, 363-8.
Holmberg, E. B., Hillman, R. E. and Perkell, J. S. (1988). ‘Glottal airflow and transglottal air pressure measurements for male and female speakers in soft, normal and loud voice’. J. Soc. Acoust. Am. 84, 511-29.
Holmberg, E. B., Hillman, R. E., Perkell, J. S., Guiod, P. C. and Goldman, S. (1995). ‘Comparisons among aerodynamic, electroglottographic, and acoustic spectral measures for female voice’. J. Speech Hear. Res. 38, 1212-23.
Junqua, J. C. (1993). ‘The Lombard reflex and its role on human listeners and automatic speech recognizers’. J. Acoust. Soc. Am. 93, 510-24.
Krause J. C. and Braida L. D. (2002). ‘Investigating alternative forms of clear speech: The effects of speaking rate and speaking mode on intelligibility’. J. Acoust. Soc. Am. 112, 2165-2172.
Ladefoged, P. (1967). ‘Three Areas of Experimental Phonetics’. Oxford U. P., London.
Liénard, J-S. and Di Benedetto, M-G. (1999). ‘Effect of vocal effort on spectral properties of vowels’. J. Acoust. Soc. Am. 106, 411-22.
Lombard, E. (1911). ‘Le Signe de l'Elevation du Voix’. Ann. Maladiers Oreille, Larynx, Nez, Pharynx 37, 101-19.
Loren, C. A., Colcord, R. D., and Rastatter, M. P. (1986). ‘Effects of auditory masking by white noise on variability of fundamental frequency during highly similar productions of spontaneous speech’. Percept. Mot. Skills 63, 1203-6.
Pearsons, K. S., Bennett, R. L. and Fidell, S. (1978). ‘Speech levels in various environments’. Bolt, Baranek and Newman Report 3281.
Rastatter, M. P. and Rivers, C. (1983). ‘The effects of short-term auditory masking on fundamental frequency variability’. J. Aud. Res. 23, 33-42.
Rostolland, D. (1982). ‘Acoustic features of shouted speech’. Acoustica 50, 118-25.
Schulman, R. (1989). ‘Articulatory dynamics of loud and normal speech’. J. Acoust. Soc. Am. 85, 295-312.
Sullivan, R. F. (1963). ‘Report on Dr. Lombard's original research on the voice reflex test’. Acta. Otolaryngol. 56, 490-2.
Summers, W. Van, Pisoni, D. B., Bernacki, R. H., Pedlow, R. I., and Stokes, M. A. (1988). ‘Effect of noise on speech production: Acoustic and perceptual analyses’. J. Acoust. Soc. Am. 84, 3, 917-28.
Södersten, M., Hertegärd, S. and Hammarberg, B. (1995). ‘Glottal closure, transglottal air-flow, and voice quality in healthy middle-aged women’. J. Voice 9, 182-97.
Traunmüller, H. and Eriksson, A. (2000). ‘Acoustic effects of variation in vocal effort by men, women, and children’. J. Acoust. Soc. Am. 107, 6, 3438-51.

1. A method of improving speech intelligibility for a listener receiving a speech signal output through a transducer in a noisy environment, where in the speech signal prior to the output one or more parameters have been modified in a signal processor corresponding to what a speaking person would normally do when speaking in a noisy environment or when speaking clearly.

2. A method according to claim 1, where at least one between the following parameters is modified: level, frequency spectrum, rate of speaking, pitch F₀, formant frequencies, F₁, F₂, . . . vowel and consonant duration, consonant/vowel energy ratio

3. A device for improving speech intelligibility for a listener receiving a speech signal output through a transducer in a noisy environment, where in the speech signal prior to the output one or more parameters have been modified in a signal processor corresponding to what a speaking person would normally do when speaking in a noisy environment or when speaking clearly.

4. A device according to claim 3, where at least one between the following parameters is modified: level, frequency spectrum, rate of speaking, pitch F₀, formant frequencies, F₁, F₂, . . . vowel and consonant duration, consonant/vowel energy ratio.
